(19) United States
(12) Patent Application Publication
(10) Pub. No.: US 2025/0057471 A1
(43) Pub. Date: Feb. 20, 2025

(54) APNEA-BRADYCARDIA-DESATURATION EVENT MONITORING

(71) Applicant: Covidien LP, Mansfield, MA (US)

(72) Inventors: Philip C. Smit, Hamilton (GB); Dean Montgomery, Edinburgh (GB); Paul S. Addison, Edinburgh (GB)

(21) Appl. No.: 18/800,727

(22) Filed: Aug. 12, 2024

Related U.S. Application Data
(60) Provisional application No. 63/520,519, filed on Aug. 18, 2023.

(51) Int. Cl. A61B 5/00; A61B 5/0205; A61B 5/024; A61B 5/08; A61B 5/091; A61B 5/1455

(52) U.S. Cl. CPC A61B 5/4818; A61B 5/0205; A61B 5/14551; A61B 5/024; A61B 5/0826; A61B 5/091; A61B 2503/04

(57) ABSTRACT

A method including determining, based on an image signal received from a camera focused on at least a portion of a patient, a breathing signal, determining an onset of a desaturation event for the patient and an onset of a bradycardia event for the patient, determining length of a bradycardia-desaturation (BD) period between the onset of the desaturation event for the patient and the onset of the bradycardia event for the patient, in response to determining that the length of the BD period is less than a BD time threshold, determining whether a change in of breathing of the patient within an apnea search period indicates an apnea event, and in response to determining that the change in of breathing of the patient within the apnea search period indicates an apnea event, displaying an occurrence of an apnea-bradycardia-desaturation event.

### CROSS-REFERENCE TO RELATED APPLICATION

The present application claims the benefit of U.S. Provisional Patent Application Ser. No. 63/520,519, filed on Aug. 18, 2023, the entire content of which is incorporated herein by reference.

Apnea is a period when breathing briefly stops, it often triggers bradycardia, which is a slow heartbeat. In neonates, apnea and bradycardia often occur together, leading to oxygen desaturation (low blood oxygen levels). Apnea-bradycardia-desaturation (ABD) events in neonatal intensive care units (NICUs) are often associated with poor outcomes and may lead to interventions by a clinician. The identification of the ABD events is important for identifying neonates at risk of poor outcomes. Furthermore, the occurrence of ABD events can play a key role in decisions made by clinicians, for example, if a neonate can be discharged.

### SUMMARY

Implementations described herein disclose a method including determining, based on an image signal received from a camera focused on at least a portion of a patient, a breathing volume signal, determining an onset of a desaturation event for the patient and an onset of a bradycardia event for the patient, determining length of a bradycardia-desaturation (BD) period between the onset of the desaturation event for the patient and the onset of the bradycardia event for the patient, in response to determining that the length of the BD period is less than a BD time threshold, determining whether a change in volume of breathing of the patient within an apnea search period indicates an apnea event, and in response to determining that the change in volume of breathing of the patient within the apnea search period indicates an apnea event, displaying an occurrence of an apnea-bradycardia-desaturation event.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Other implementations are also described and recited herein.

### BRIEF DESCRIPTION OF THE DRAWINGS

A further understanding of the nature and advantages of the present technology may be realized by reference to the figures, which are described in the remaining portion of the specification.

FIG. 1 illustrates an example implementation of an Apnea-Bradycardia-Desaturation (ABD) event monitoring system disclosed herein.

FIG. 2 indicates an example graph illustrating signals from a depth sensing camera in conjunction with other physiological parameter signals.

FIG. 3 illustrates example operations of the ABD event monitoring system disclosed herein.

FIG. 4 illustrates alternative example operations of the ABD event monitoring system disclosed herein.

FIG. 5 illustrates an alternative example graph illustrating signals from a depth sensing camera in conjunction with other physiological parameter signals.

FIG. 6 shows a portable non-contact subject monitoring system that includes a non-contact detector and a computing device.

FIG. 7 shows a semi-portable non-contact subject monitoring system that includes a non-contact detector and a computing device.

FIG. 8 shows a non-portable non-contact subject monitoring system that includes a non-contact detector and a computing device.

FIG. 9 is a block diagram illustrating a system including a computing device, a server, and an image capture device.

### DETAILED DESCRIPTIONS

Apnea-bradycardia-desaturation (ABD) events in neonatal intensive care units (NICUs) are often associated with poor outcomes and may lead to interventions by a clinician. The identification of the ABD events is important for identifying neonates at risk of poor outcomes. Furthermore, the occurrence of ABD events can play a key role in decisions made by clinicians, for example, if a neonate can be discharged. In the NICU, the heart rate and oxygen saturation may be continuously monitored with high fidelity and bradycardia and desaturation may be identified using signals derived from attached probes. However, due to the poor quality of respiratory monitoring currently, apneas are often identified manually. This may lead to a semi-automated and time-consuming process where manual interpretation of apnea events may lead to inter-user variability.

The technology disclosed herein uses a depth-sensing vision system to properly identify ABD events. When combined with signals from a pulse oximeter, the system disclosed herein fully automates ABD monitoring. Specifically, the ABD monitoring system disclosed herein generates a breathing volume signal based on an image signal received from a camera focused on at least a portion of a patient. The ABD monitoring system also determines an onset of a desaturation event for the patient and an onset of a bradycardia event for the patient. The ABD monitoring system may monitor heart rate (HR) and/or oxygen saturation (SpO₂) signal to determine a time period during which an ABD event, an apnea-bradycardia (AB) event, or an apnea-desaturation (AD) event may have occurred. Subsequently, one or more physiological signals during this time period are further analyzed to determine existence of such events.

Typically, the breathing volume signal of a patient is more complex and noisier than the bradycardia signal and/or the desaturation signal. Therefore, it may be more difficult to analyze the breathing volume signal of the patient to determine an apnea event compared to analyzing the bradycardia signal and/or the desaturation signal. The implementations disclosed herein identifies an onset of a bradycardia event and/or an onset of a desaturation event before considering the breathing volume signal. Specifically, an implementation evaluates a period between the onset of a bradycardia event and the onset of a desaturation event, referred to as a BD period, and if the BD period is below a bradycardia-desaturation (BD) time threshold, it analyzes the breathing volume signal to determine presence of an apnea event. Such implementations allow the algorithm for detecting an apnea event to be more effective in correctly detecting apnea than if the breathing volume signal were evaluated without the knowledge of the bradycardia event and/or the desaturation event.

Evaluating the breathing volume signal to determine apnea event without consideration of a bradycardia event and/or a desaturation event may result in more false positives due to the complex nature of the breathing volume signal. By limiting the search of an apnea event to time periods around the onset of the bradycardia event and the desaturation event, the number of such false positives are minimized. Furthermore, the BD period below the BD time threshold indicates close occurrence of the bradycardia event and the desaturation event, which further allows narrowing down the time period during which the breathing volume signal may be analyzed to detect an apnea event with lower probability of false positive.

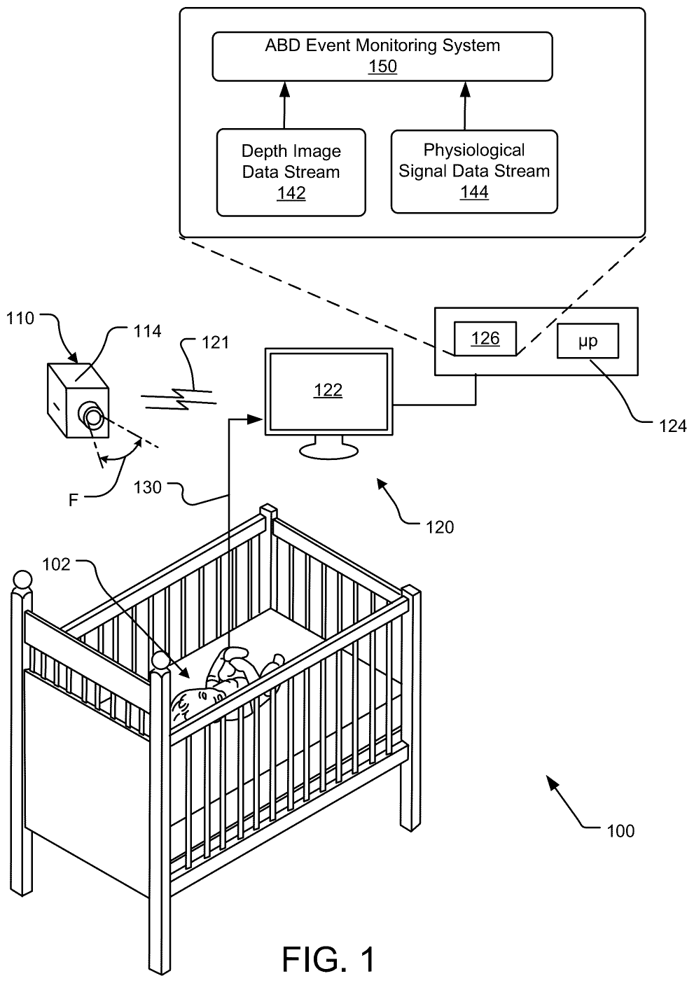

FIG. 1 illustrates a non-contact subject monitoring system 100 for a subject 102, in this particular example an infant in a crib. It is noted that the systems and methods described herein are not limited to a crib, but may be used with a bassinette, an incubator, an isolette, or any other place where the subject 102 is. The system 100 includes a non-contact detector system 110 placed remote from the subject 102. In this embodiment, the detector system 110 includes a camera system 114, particularly, a camera that may include an infrared (IR) detection feature. The camera system 114 may be a depth sensing camera system, such as a Kinect camera from Microsoft Corp. (Redmond, Washington) or a RealSense™ D415, D435 or D455 camera from Intel Corp. (Santa Clara, California).

The camera system 114 is remote from the subject 102, in that it is spaced apart from and does not physically contact the subject 102. The camera system 114 may be positioned in close proximity to or on the crib. The camera system 114 has a field of view F that encompasses at least a portion of the subject 102. The field of view F may be selected to be at least the upper torso of the subject 102. However, as it is common for young children and infants to move within the confines of their crib, bed or other sleeping area, the entire area potentially occupied by the subject 102 (e.g., the crib) may be the field of view F.

The camera system 114 includes a depth sensing camera that can detect a distance between the camera system 114 and objects in its field of view F. Such information can be used to determine that the subject 102 is within the field of view of the camera system 114 and determine a region of interest (ROI) to monitor on the subject. The ROI may be the entire field of view For may be less than the entire field of view F. Once an ROI is identified, the distance to the desired feature is determined and the desired measurement(s) can be made.

The measurements (e.g., one or more of depth signal, RGB reflection, light intensity) are sent to a computing device 120 through a wired or wireless connection 121. The computing device 120 includes a display 122, a processor 124, and hardware memory 126 for storing data, software, computer instructions, etc. Sequential image frames of the subject 102 are recorded by the video camera system 114 and sent to the computing device 120 for analysis by the processor 124. The display 122 may be remote from the computing device 120, such as a video screen positioned separately from the processor and memory. Other embodiments of the computing device 120 may have different, fewer, or additional components than shown in FIG. 1. In some embodiments, the computing device 120 may be a server. In other embodiments, the computing device 120 of FIG. 1 may be connected to a server. The captured images (e.g., still images or video) can be processed or analyzed at the computing device 120 and/or at the server to create a topographical map or image to identify the subject 102 and any other objects within the ROI.

Also in some embodiments, the computing device 120 is communicatively connected to a monitoring device (not shown) that collects one or more physiological signals 130 from the patient 120. For example, such physiological signals may include the patient oxygen saturation rate SpO₂ from an oximeter, the patient heart rate (HR) from an electrocardiogram (ECG) monitor, the patient respiration rate, etc. For example, the subject 102 may be connected to other physiological signal probes to generate the physiological signals 130 that are communicated to the computing device 120. For example, the subject 102 may be connected to an electrocardiogram (ECG) probe that generates heart rate, an oxygen saturation probe that generates SpO₂ signal, and a transthoracic electrocardiogramanerator that generates transthoracic impendence (TTI) signal (together referred to as the physiological signals 130).

The signals collected from the camera system 114 and the physiological signals 130 may be stored in the memory 126. For example, the signals from the camera system 114 may be stored as a depth image data stream 142 and the physiological signals 130 may be stored as a physiological signal data stream 144. Furthermore, the memory 126 may store various computer programs, software, instructions, etc., to process the data including the depth image data stream 142 and the physiological signal data stream 144.

For example, the memory 126 may store various instructions for an apnea-bradycardia-desaturation (ABD) event monitoring system 150. The ABD monitoring system 150 may include various modules to process the depth image data stream 142 to generate respiratory signal or breathing volume signal (together referred to herein as the “breathing volume signal”) of the subject 102. For example, the breathing volume signal of the subject 102 may indicate the number of breathes per second by the subject 102 as well as the volume of air breathed in by the subject 102 per second. In one implementation, such breathing volume signal is generated by analyzing the depth image data stream 142 where depth of frames of signals from the camera system 114 are analyzed and the changes in the depth of such frames together with the width of a portion of the subject 102 experiencing such changes in the depth are used to calculate the rate of breathing and the volume of air breathed by the subject.

Subsequently, the breathing volume signal is analyzed to determine an apnea event. For example, a prolonged absence of breathing, as indicated by the breathing volume signal having lower amplitude for the prolonged period may indicate such an apnea event. In one implementation, such absence of breathing for ten (10) seconds or more may be considered to be indicative of an apnea event. As a result of the apnea, the level of oxygen in the subject 102 may fall below normal levels, resulting in low oxygen saturation levels indicated by the SpO₂ levels. As an example, a decrease in the level of oxygen saturation below 80% may be indicative of a desaturation event. Similarly, the reduced level of breathing may also lead to reduced heart rate for the subject 102. For example, a heart rate dropping below 100 bpm for the subject may indicate a bradycardia event.

It is important to monitor the occurrence of an apnea event, which is often followed by a bradycardia event and a desaturation event. Occurrence of apnea, bradycardia, and/or desaturation (ABD) may be a critical event for the health of the subject 102. In one implementation, an ABD event may be defined when the subject 102, which may be a neonate, exhibits the following symptoms:
an apnea for >10 seconds
a heart rate HR<100 bpm
an oxygen saturation SpO₂<80%

However, in an alternative implementation, combination of different threshold levels may be used to indicate occurrence of an ABD event. For example, in another implementation, an ABD event may be defined when the subject 102, which may be a neonate, exhibits the following symptoms:
an apnea for >20 seconds
a heart rate HR<80 bpm
an oxygen saturation SpO₂<90%

Yet alternatively, in another implementation, an ABD is determined to have occurred when one or more of the above thresholds are held for more than predetermined time periods. Similarly, a combination of apnea followed by bradycardia (AB event), or apnea followed by desaturation (AD event) may also be critical to the health of the subject 102. Therefore, the ABD event monitoring system 150 may be configured to also monitor occurrences of such AB events and AD events.

Additionally, the ABD event monitoring system 150 may also first determine an existence of a BD event indicating a combination of the heart rate below an HR threshold and the oxygen saturation levels below an SpO₂ threshold. Subsequently, the ABD event monitoring system 150 defines a period of time before and after the occurrence of the BD event to evaluate the depth image data stream 142 to see if an apnea event has occurred during this period. For example, suppose that the heart rate drops below the HR threshold at T_{B1} and the oxygen saturation levels below an SpO₂ threshold T_{D1}. Note that T_{B1} and T_{D1} may occur in any order. In this case, the ABD event monitoring system 150 may determine that if the absolute time difference between T_{B1} and T_{D1} is less than a BD time threshold T_{BD}, a BD event has occurred. For example, the BD time threshold T_{BD} time may be ten (10) seconds. In this case:

$$ABS ( {T}_{B 1} - {T}_{D 1} ) <= {T}_{BD} \to BD event$$

where ABS represents an absolute value function.

Once the BD event is identified, the ABD event monitoring system 150 may search the depth image data stream 142 over a time period W1 backwards from the earlier of T_{D1} and T_{B1} and over a period W2 forwards from the later of T_{D1} and T_{B1}. If an apnea event is located within an apnea search period of length=W1+ABS (T_{B1}−T_{D1})+W2, then this region may be classified as containing an ABD event.

In one implementation, the ABD event monitoring system 150 may analyze the depth image data stream 142 over the apnea search period and if it finds a period over which the depth image data stream 142 indicates breath modulations of amplitude over a number of breaths less than a preset fraction “K” of the breath amplitude A_{RR} for breaths over a preceding period, the ABD event monitoring system 150 may determine this to be an apnea event. For example, such preset fraction may be 20%. Thus, if for a period within the apnea search period, the modulation of breath has an average amplitude that is 20% below the average breath amplitudes A_{RR} over a preceding period, for say over a predetermined period of ten seconds, an occurrence of apnea maybe indicated.

Thus, the ABD event monitoring system 150 may analyze the breathing volume signal, generated based on the depth image data stream 142, over a first section of the apnea search period to calculate a present breathing volume, analyze the breathing volume signal over a preceding section of the apnea search period to calculate a threshold breathing volume, the preceding section being before the first section, and determine that the change in volume of breathing of the patient within the apnea search window indicates an apnea event when the present breathing volume is below a fraction K of the threshold breathing volume.

Alternatively, instead of using the relative drop in the respiratory signal to determine an apnea event, the ABD event monitoring system 150 may determine an apnea to have occurred if the breathing volume indicated by the respiratory signal is less than an absolute breathing volume e.g., 1 ml, 2 ml, 5 ml, 10 ml, 20 ml, 50 ml. In one implementation, such absolute breathing volume may be dependent upon the size or age of the subject 102. For example, the absolute breathing volume may be lower for a smaller patient compared to the absolute breathing volume for a larger patient. Similarly, the absolute breathing volume may be lower for a younger patient compared to the absolute breathing volume for an older patient.

Thus, the ABD event monitoring system 150 may analyze the breathing volume signal, generated based on the depth image data stream 142, over a first section of the apnea search period to calculate a present breathing volume, compare the present breathing volume to an absolute breathing volume for the patient, and in response to determining that that present breathing volume is below the absolute breathing volume for the patient, determine occurrence of the apnea event.

Furthermore, to remove any noise in the depth image data stream 142 due to, for example, movements by the subject 102, the ABD event monitoring system 150 may detect individual spikes in breathing volume signals and discount it from the breath amplitude search. Similarly, to remove any noise in the depth image data stream 142 due to the periods when the subject 102 maybe moving but not breathing, the ABD event monitoring system 150 may detect motion from the depth signal where there may be lack of breathing volume and include this period within the apnea search period.

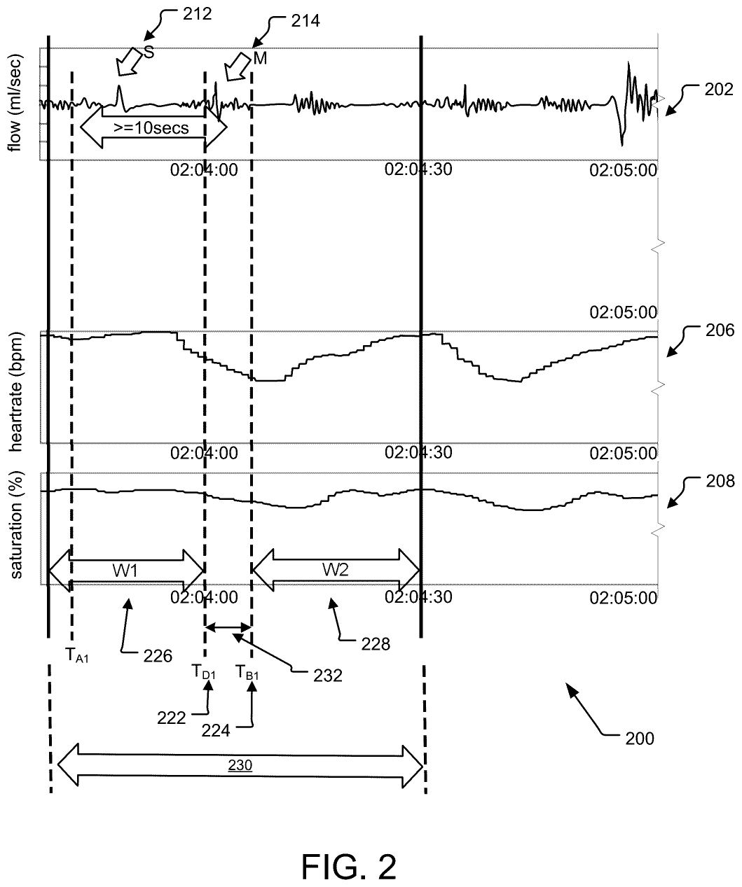

FIG. 2 indicates a graph 200 illustrating breathing signals derived from depth signals from a depth sensing camera in conjunction with other physiological parameter signals. Specifically, the graph 200 includes a breathing signal 202 indicating flow of breathing for a patient in ml/sec. Specifically, the breathing signal 202 may be derived from an image signal and/or depth signal received from a camera that is focused on a part of a body of a patient. The breathing signal 202 may indicate breathing flow by the patient in terms of milliliters (ml) of air breathed per second (ml/sec). For example, a camera focused on a patient may generate a sequence of depth images including the depth information within a field of view. The sequence of depth images generated by the camera are communicated to a computing system that analyzes the sequence of depth images to generate a breathing signal associated with the breathing by the patient. For example, such flow signals associated with the breathing by the patient may be generated based on the change in the depth of the chest and/or abdominal regions of the patient.

The graph 200 also includes a heart rate (HR) signal 206 indicating the heart rate of the patient and an SpO₂ signal 208 indicating oxygen saturation level for the patient. For example, the HR signal 206 may be generated by a heart rate monitor and the SpO₂ signal may be generated by a pulse oximeter. Specifically, the graph 200 indicates the signals 202-208 when the patient may be potentially experiencing an ABD event. Specifically, as indicated by the HR signal 206, the heart rate of the patient may have dropped below a HR threshold at time T_{B1} 224 indicating a bradycardia (B) event. Similarly, the SpO₂ signal 208 indicates that the oxygen saturation level of the patient may have dropped below an SpO₂ threshold level at T_{D1} 222 indicating a desaturation (D) event. As the HR signal 206 and the SpO₂ signal 208 are typically lagging indicators of an ABD event, in that they follow onset of an apnea (A) event, the breathing signal 202 maybe analyzed around T_{B1} and T_{D1} to determine existence of an apnea event. Note that T_{B1} and T_{D1} may happen in any order, before, during, or following an apnea event.

In the illustrated example, an ABD event monitoring system, such as the ABD event monitoring system 150 disclosed in FIG. 1, evaluates an apnea search period 230 that includes a preceding period W1 226, a following period W2 228, and the period ABS (T_{B1}−T_{D1}), wherein the ABS indicates an absolute value function. The period between T_{B1} and T_{D1} may be referred to as the BD period 232. In the illustrated implementation, the apnea search period 230 may begin before the BD period 232 and ending after the BD period 232. Specifically, the preceding period W1 226 terminates at the earlier of T_{B1} and T_{D1}, whereas the following period W2 228 starts at the later of T_{B1} and T_{D1}. Thus, the apnea search period 230 may be of a length W1+ABS (T_{B1}−T_{D1})+W2.

However, in an alternate implementation, the apnea search period 230 may begin at a different point in time with respect to the BD period 232. For example, the apnea search period 230 may begin at earlier of the T_{B1} and T_{D1} and end at the later of the T_{B1}-T_{D1}. Alternatively, the apnea search period 230 may begin before the earlier of the T_{B1} and T_{D1} and end at the later of the T_{B1} and T_{D1}. Yet alternatively, the apnea search period 230 may begin at the earlier of the T_{B1} and T_{D1} and end after the later of the T_{B1} and T_{D1}. In any of these implementations, because the location and the length of the apnea search period 230 are determined in the context of T_{B1} and T_{D1}, the chances of finding a false positive for an apnea event are minimized.

In one implementation, the ABD event monitoring system may analyze the breathing signal 202 over the apnea search period 230 and if it finds a period over which the breathing signal 202 indicates breath modulations of amplitude over a number of breaths less than a preset fraction “K” of an average amplitude A_{RR} of the preceding breaths, the ABD event monitoring system may determine this to be an apnea event. For example, such preset fraction may be 20%. Alternatively, instead of the using the relative drop in the breathing signal 202 to determine an apnea event, the ABD event monitoring system may determine an apnea to have occurred if the breathing signal 202 is less than an absolute flow threshold e.g., 1 ml/sec, 2 ml/sec, 5 ml/sec, 10 ml/sec, 20 ml/sec, 50 ml/sec. For example, such threshold maybe dependent upon the size or age of the subject.

While the above disclosed implementation compares relative drop in breathing signal 202, where the breathing signal 202 indicates breathing flow, to determine an apnea event, in an alternative implementation other measure of the subject's breathing signal 202 may be evaluated to determine an onset of an apnea event. For example, the volume of breathing measured in ml, may be evaluated to determine an onset of apnea. In such implementation, the volume of breathing may be compared to a volume threshold of, e.g., 1 ml, 2 ml, 5 ml, 10 ml, 20 ml, etc., to determine if an apnea event has occurred. Furthermore, such volume threshold may also be dependent upon the size of the subject and/or on the age of the subject.

Furthermore, to remove any noise in the breathing signal 202 due to, for example, movements by the subject, the ABD event monitoring system may detect individual spikes in the breathing signal 202 such as a spike S 212 and discount it from the breath amplitude. Similarly, to remove any noise in the breathing signal 202 due to the periods when the subject maybe moving but not breathing as indicated by M 214, the ABD event monitoring system may detect such motion based on the depth signals during a period with low breathing signal 202 and include this period within the apnea search period 230.

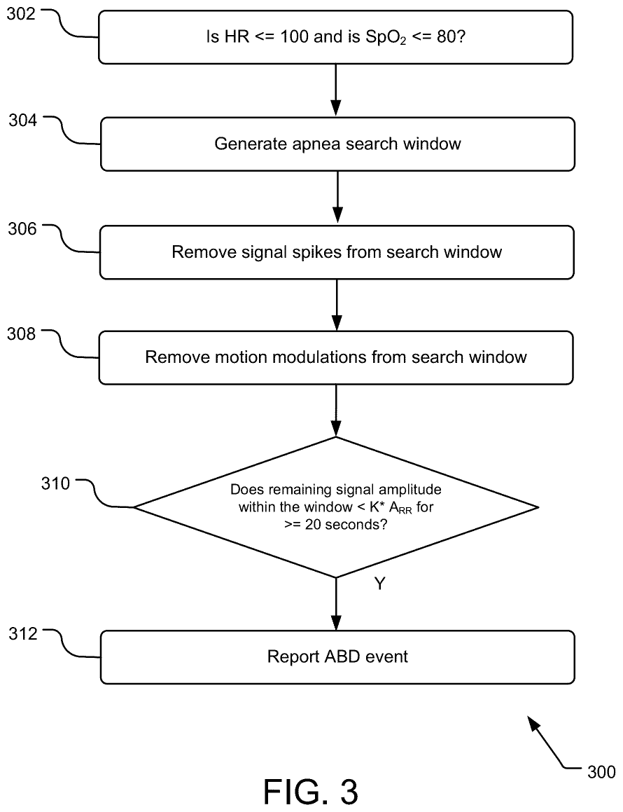

FIG. 3 illustrates operations 300 of the ABD event monitoring system disclosed herein. Specifically, an operation 302 determines if there is an occurrence of a BD event. For example, in one implementation, if the heart rate is below 100 bpm, it may be construed to indicate a bradycardia (B) event and if the SpO₂ is below 80%, it may be construed to indicate a desaturation (D) event. While, in the illustrated implementation, the operations 300 are used to detect an ABD event, if the operations 300 are used to detect an occurrence of an AB event, the operation 302 only determines if the heart rate is below 100 bpm. On the other hand, if the operations 300 are used to detect an occurrence of an AD event, the operation 302 only determines if SpO₂ is below 80%.

Subsequently, an operation 304 generates an apnea search period. In one implementation, the apnea search period may include a preceding period W1 terminating at the earlier of the bradycardia (B) event and the desaturation (D) event, a following period W2 beginning at the later of the bradycardia (B) event and the desaturation (D) event, and the period between the bradycardia (B) event and the desaturation (D) event. Thus, the apnea search period may be defined to have a length of W1+ABS (T_{B1}−T_{D1})+W2.

An operation 306 removes any local spikes in the breathing volume signal within the apnea search period. For example, such local spike in the breathing volume signal may be due to the patient's movement. The operation 306 may compare the amplitude of the breathing volume signal with an amplitude threshold to see if there are any spikes indicating breathing volume signal amplitude above such amplitude threshold. An operation 308 removes any motion noise when the patient may be moving but not breathing. Specifically, the operation 308 may detect the motion of the patient by analyzing the depth images from a camera during a period within the apnea search period where the breathing volume signal indicates that the patient is not breathing.

Once the breathing volume signal within the apnea search period is cleaned of signal spikes and/or patient motion modulations, an operation 310 determines if an apnea event has occurred. For example, the operation 310 calculates modulations of amplitude over periods of predetermined width and compares the amplitude with an average amplitude of breaths in preceding period A_{RR}. If the amplitude over a period drops by a predetermined percentage K below the average amplitude of breaths in preceding period A_{RR} and stays below this for a length of time, the operation 310 determines it to be an apnea event. For example, the K may be 20%, in which case if the breathing volume amplitude over a given section falls by 20% below the of breaths in preceding period A_{RR} for more than ten (10) seconds, the operation 310 may determine an apnea event. In response to determining an occurrence of an apnea event in the apnea search period, an operation 312 reports an ABD event to a patient monitoring system. Alternatively, the operation 310 may use alternative period such as thirty (30) seconds, twenty (20) seconds, five (5) seconds, etc., to determine an apnea event.

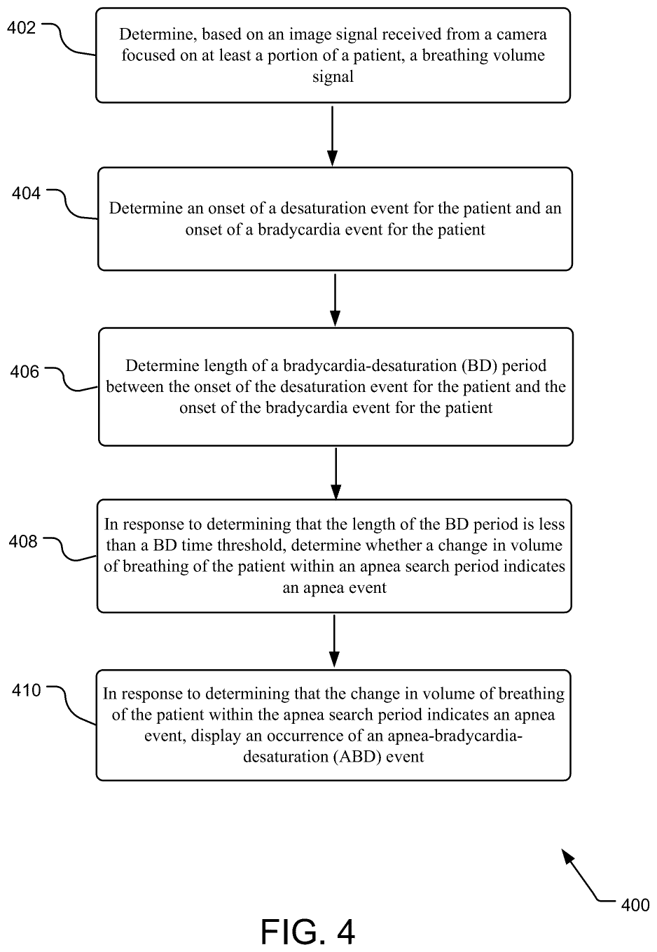

FIG. 4 illustrates alternative operations 400 of the ABD event monitoring system disclosed herein. An operation 402 determines, based on an image signal received from a camera focused on at least a portion of a patient, a breathing signal. Subsequently, an operation 404 determines an onset of a desaturation event for the patient and an onset of a bradycardia event for the patient. An operation 406 determines length of a bradycardia-desaturation (BD) period between the onset of the desaturation event for the patient and the onset of the bradycardia event for the patient. Specifically, the period between the onset of bradycardia at TB₁ and the onset of desaturation at TD₁ is referred to as the BD period. The BD period may be given by ABS (TB₁−TD₁).

An operation 408 determines that the length of the BD period is less than a BD time threshold T_{BD}, wherein T_{BD} is a bradycardia-desaturation (BD) time threshold, and in response determines whether a change of breathing of the patient within an apnea search period indicates an apnea event. Here the apnea search period may be of length W1+ABS (TB₁−TD₁)+W2, wherein W1 is a period preceding the earlier of TB₁ and TD₁ and W2 is a period following the later of TB₁ and TD₁. In one implementation, the BD time threshold T_{BD} may be ten (10) seconds, however, alternative BD time thresholds T_{BD} may also be used.

Subsequently, in response to determining that the change in breathing of the patient within the apnea search period indicates an apnea event, an operation 410 displays an occurrence of an apnea-bradycardia-desaturation (ABD) event.

Typically, the breathing volume signal is more complex and noisier than the bradycardia signal and/or the desaturation signal. Therefore, it may be more difficult to analyze the breathing volume signal of the patient to determine an apnea event compared to analyzing the bradycardia signal and/or the desaturation signal. The implementations disclosed herein identifies an onset of a bradycardia event and/or an onset of a desaturation event before considering the breathing volume signal. Specifically, the implementations evaluate the period between the onset of a bradycardia event and the onset of a desaturation event, and if such period is below the BD time threshold, it analyzes the breathing volume signal to determine presence of an apnea event. Such implementations allow the algorithm for detecting an apnea event to be more effective in correctly detecting apnea than if the breathing volume signal were evaluated without the knowledge of the bradycardia event and/or the desaturation event.

Specifically, evaluating the breathing volume signal to determine apnea event without consideration of a bradycardia event and/or a desaturation event may result in more false positives due to the complex nature of the breathing volume signal. By limiting the search of an apnea event to time periods around the onset of the bradycardia event and the desaturation event, the number of such false positives are minimized. Furthermore, the BD period below the BD time threshold indicates close occurrence of the bradycardia event and the desaturation event, which further allows narrowing down the time period during which the breathing volume signal may be analyzed to detect an apnea event with lower probability of false positive.

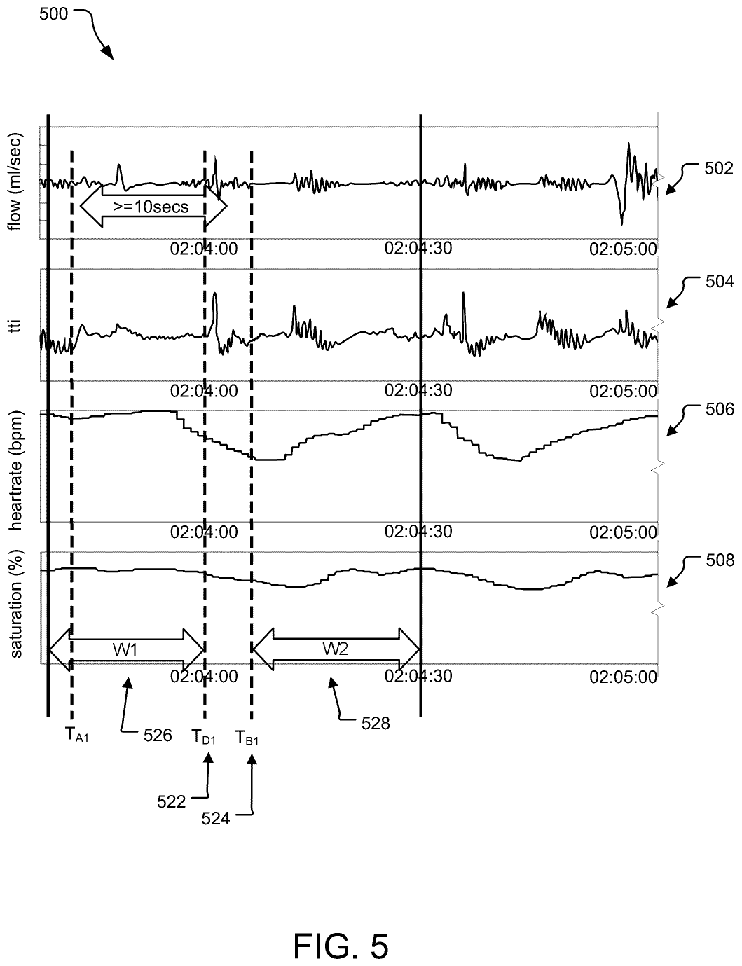

FIG. 5 illustrates an alternative graph 500 illustrating signals from a depth sensing camera in conjunction with other physiological parameter signals. Specifically, the graph 500 includes a breathing volume signal 502 indicating a breathing volume for a patient, a transthoracic impendence (TTI) signal 504, a heart rate (HR) signal 506 indicating the heart rate of the patient, and an SpO₂ signal 508 indicating oxygen saturation level for the patient. Specifically, as illustrated an apnea event that can be determined by analysis of the breathing volume signal 502 may also be determined alternatively by analysis of the TTI signal 504.

Thus, in response to determining a bradycardia (B) event at TB₁ 524 or a desaturation (D) event at TD₁ 522, the TTI signal 504 may be evaluated over an apnea search period including a preceding period W1 526 preceding the earlier of TD₁ and TB₁, a following period W2 528 following the later of TD₁ and TB₁, and the period between TD₁ and TB₁.

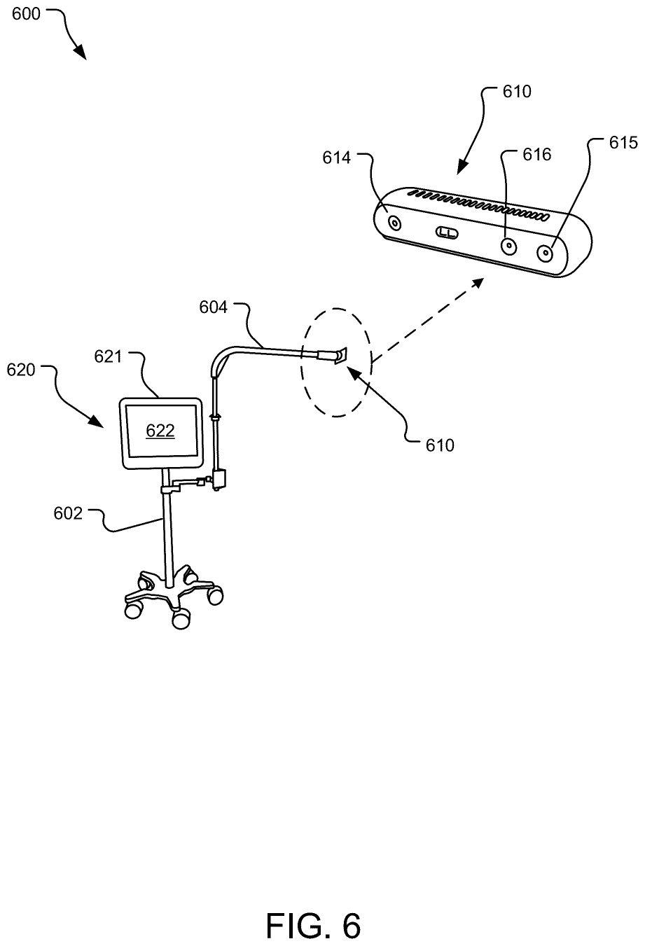

FIG. 6 shows a portable non-contact subject monitoring system 600 that includes a non-contact detector 610 and a computing device 620. In this embodiment, the non-contact detector 610 and the computing device 620 are generally fixed in relation to each other and the system 600 is readily moveable in relation to the subject to be monitored. The detector 610 and the computing device 620 are supported on a trolley or stand 602, with the detector 610 on an arm 604 that is pivotable in relation to the stand 602 as well as adjustable in height. The system 600 can be readily moved and positioned where desired.

The detector 610 includes a first camera 614 and a second camera 615, at least one of which includes an infrared (IR) camera feature. The detector 610 also includes an IR projector 616, which projects individual features (e.g., dots, crosses or Xs, lines, or a featureless pattern, or a combination thereof etc.).

The detector 610 may be wired or wireless connected to the computing device 620. The computing device 620 includes a housing 621 with a touch screen display 622, a processor (not seen), and hardware memory (not seen) for storing software and computer instructions.

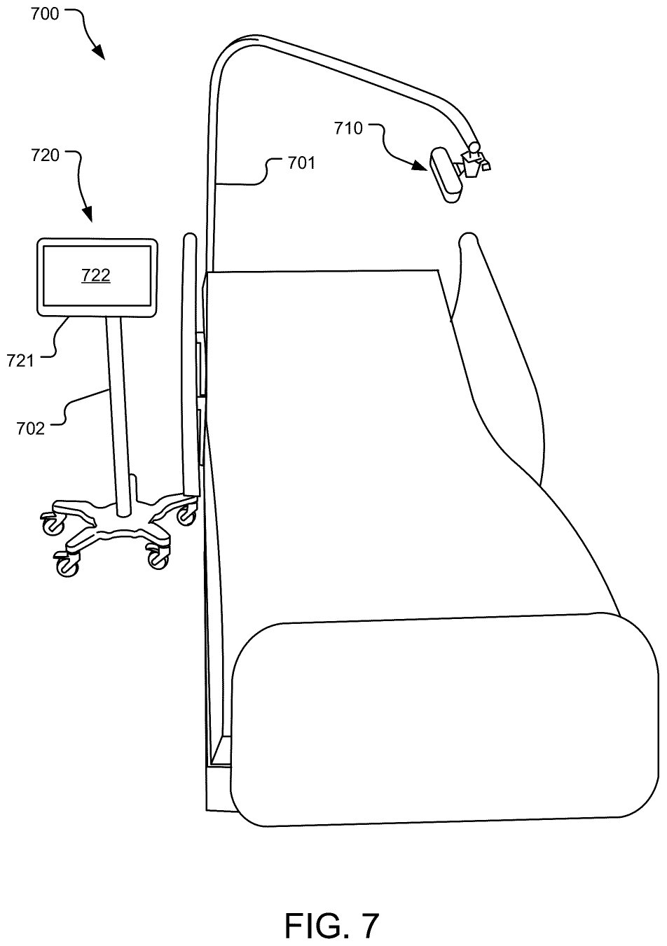

FIG. 7 shows a semi-portable non-contact subject monitoring system 700 that includes a non-contact detector 710 and a computing device 720. In this embodiment, the non-contact detector 710 is in a fixed relation to the subject to be monitored and the computing device 720 is readily moveable in relation to the subject.

The detector 710 is supported on an arm 701 that is attached to a bed, in this embodiment, a hospital bed, although the detector 710 and the arm 701 can be attached to a crib, a bassinette, an incubator, an isolette, or other bed-type structure. In some embodiments, the arm 701 is pivotable in relation to the bed as well as adjustable in height to provide for proper positioning of the detector 710 in relation to the subject.

The detector 710 may be wired or wireless connected to the computing device 720, which is supported on a moveable trolley or stand 702. The computing device 720 includes a housing 721 with a touch screen display 722, a processor (not seen), and hardware memory (not seen) for storing software and computer instructions.

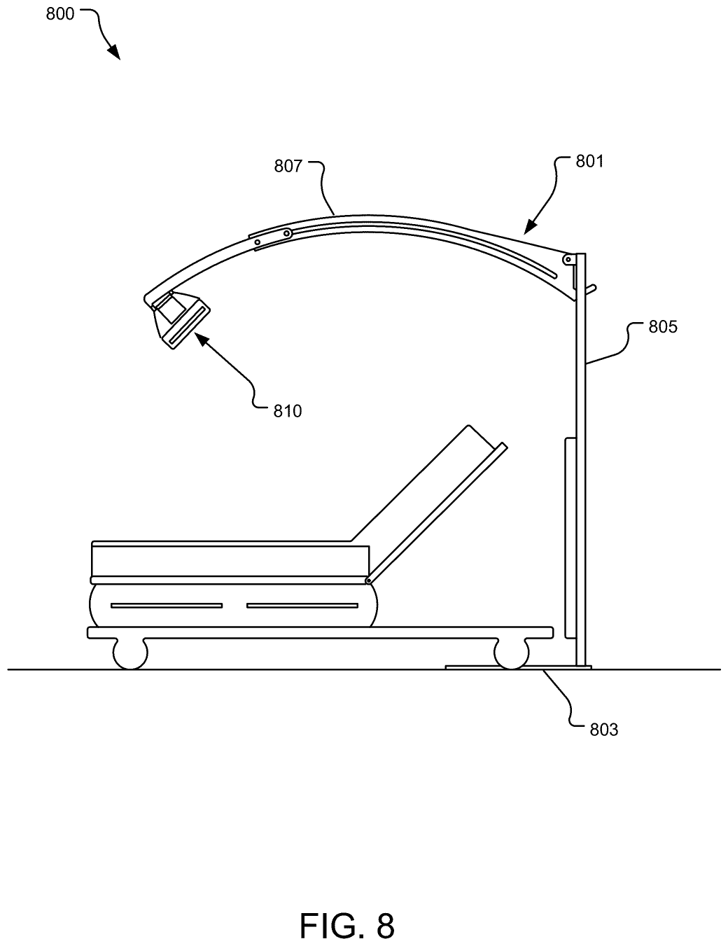

FIG. 8 shows a non-portable non-contact subject monitoring system 800 that includes a non-contact detector 810 and a computing device (not seen in FIG. 8). In this embodiment, at least the non-contact detector 810 is generally fixed in a location, configured to have the subject to be monitored moved into the appropriate position to be monitored.

The detector 810 is supported on a stand 801 that is free standing, the stand having a base 803, a frame 805, and a gantry 807. The gantry 807 may have an adjustable height, e.g., movable vertically along the frame 805, and may be pivotable, extendible and/or retractable in relation to the frame 805. The stand 801 is shaped and sized to allow a bed or bed-type structure to be moved (e.g., rolled) under the detector 810.

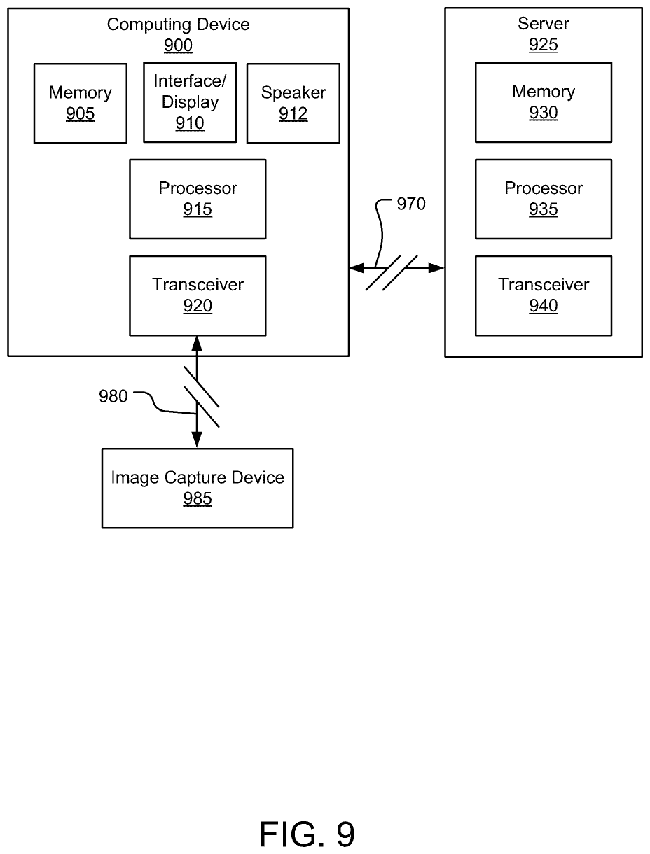

FIG. 9 is a block diagram illustrating a system including a computing device 900, a server 925, and an image capture device 985 (e.g., a camera, e.g., the camera system 114 or cameras 214, 215). In various embodiments, fewer, additional and/or different components may be used in the system.

The computing device 900 includes a processor 915 that is coupled to a memory 905. The processor 915 can store and recall data and applications in the memory 905, including applications that process information and send commands/signals according to any of the methods disclosed herein. The processor 915 may also display objects, applications, data, etc. on an interface/display 910 and/or provide an audible alert via a speaker 912. The processor 915 may also or alternately receive inputs through the interface/display 910. The processor 915 is also coupled to a transceiver 920. With this configuration, the processor 915, and subsequently the computing device 900, can communicate with other devices, such as the server 925 through a connection 970 and the image capture device 985 through a connection 980. For example, the computing device 900 may send to the server 925 information determined about a subject from images captured by the image capture device 985, such as depth information of a subject or object in an image.

The server 925 also includes a processor 935 that is coupled to a memory 930 and to a transceiver 940. The processor 935 can store and recall data and applications in the memory 930. With this configuration, the processor 935, and subsequently the server 925, can communicate with other devices, such as the computing device 900 through the connection 970.

The computing device 900 may be, e.g., the computing device 120 of FIG. 1 or the computing device 220 of FIG. 2. Accordingly, the computing device 900 may be located remotely from the image capture device 985, or it may be local and close to the image capture device 985 (e.g., in the same room). The processor 915 of the computing device 900 may perform any or all of the various steps disclosed herein. In other embodiments, the steps may be performed on a processor 935 of the server 925. In some embodiments, the various steps and methods disclosed herein may be performed by both of the processors 915 and 935. In some embodiments, certain steps may be performed by the processor 915 while others are performed by the processor 935. In some embodiments, information determined by the processor 915 may be sent to the server 925 for storage and/or further processing.

The devices shown in the illustrative embodiment may be utilized in various ways. For example, either or both of the connections 970, 980 may be varied. For example, either or both the connections 970, 980 may be a hard-wired connection. A hard-wired connection may involve connecting the devices through a USB (universal serial bus) port, serial port, parallel port, or other type of wired connection to facilitate the transfer of data and information between a processor of a device and a second processor of a second device. In another example, one or both of the connections 970, 980 may be a dock where one device may plug into another device. As another example, one or both of the connections 970, 980 may be a wireless connection. These connections may be any sort of wireless connection, including, but not limited to, Bluetooth connectivity, Wi-Fi connectivity, infrared, visible light, radio frequency (RF) signals, or other wireless protocols/methods. For example, other possible modes of wireless communication may include near-field communications, such as passive radio-frequency identification (RFID) and active RFID technologies. RFID and similar near-field communications may allow the various devices to communicate in short range when they are placed proximate to one another. In yet another example, the various devices may connect through an internet (or other network) connection. That is, one or both of the connections 970, 980 may represent several different computing devices and network components that allow the various devices to communicate through the internet, either through a hard-wired or wireless connection. One or both of the connections 970, 980 may also be a combination of several modes of connection.

The configuration of the devices in FIG. 9 is merely one physical system on which the disclosed embodiments may be executed. Other configurations of the devices shown may exist to practice the disclosed embodiments. Further, configurations of additional or fewer devices than the ones shown in FIG. 9 may exist to practice the disclosed embodiments. Additionally, the devices shown in FIG. 9 may be combined to allow for fewer devices than shown or separated such that more than the three devices exist in a system. It will be appreciated that many various combinations of computing devices may execute the methods and systems disclosed herein. Examples of such computing devices may include other types of infrared cameras/detectors, night vision cameras/detectors, other types of cameras, radio frequency transmitters/receivers, smart phones, personal computers, servers, laptop computers, tablets, RFID enabled devices, or any combinations of such devices.

In contrast to tangible computer-readable storage media, intangible computer-readable communication signals may embody computer readable instructions, data structures, program modules or other data resident in a modulated data signal, such as a carrier wave or other signal transport mechanism. The term “modulated data signal” means a signal that has one or more of its characteristics set or changed in such a manner as to encode information in the signal. By way of example, and not limitation, intangible communication signals include wired media such as a wired network or direct-wired connection, and wireless media such as acoustic, RF, infrared and other wireless media.

The implementations described herein are implemented as logical steps in one or more computer systems. The logical operations may be implemented (1) as a sequence of processor-implemented steps executing in one or more computer systems and (2) as interconnected machine or circuit modules within one or more computer systems. The implementation is a matter of choice, dependent on the performance requirements of the computer system being utilized. Accordingly, the logical operations making up the implementations described herein are referred to variously as operations, steps, objects, or modules. Furthermore, it should be understood that logical operations may be performed in any order, unless explicitly claimed otherwise or a specific order is inherently necessitated by the claim language.

The above specification, examples, and data provide a complete description of the structure and use of exemplary embodiments of the invention. Since many implementations of the invention can be made without departing from the spirit and scope of the invention, the invention resides in the claims hereinafter appended. Furthermore, structural features of the different embodiments may be combined in yet another implementation without departing from the recited claims.

What is claimed is:

1. A method, comprising:
determining, based on an image signal received from a camera focused on at least a portion of a patient, a breathing signal;
determining an onset of a desaturation event for the patient and an onset of a bradycardia event for the patient;
determining length of a bradycardia-desaturation (BD) period between the onset of the desaturation event for the patient and the onset of the bradycardia event for the patient;
in response to determining that the length of the BD period is less than a BD time threshold, determining whether a change in breathing of the patient within an apnea search period indicates an apnea event; and
in response to determining that the change in breathing of the patient within the apnea search period indicates an apnea event, displaying an occurrence of an apnea-bradycardia-desaturation (ABD) event.

2. The method of claim 1, wherein the apnea search period beginning before the BD period.

3. The method of claim 1, wherein determining the onset of the bradycardia event for the patient comprising determining at least one of whether the oxygen saturation level of the patient is below 80% and whether the heart-rate signal of the patient is below 100 bpm.

4. The method of claim 1, wherein the breathing signal is a breathing volume signal and wherein determining whether the change in breathing of the patient within the apnea search period indicates an apnea event comprises:
analyzing the breathing volume signal over a first section of the apnea search period to calculate a present breathing volume;
analyzing the breathing volume signal over a preceding section of the apnea search period to calculate a threshold breathing volume, the preceding section being before the first section; and
determining that a change in volume of breathing of the patient within the apnea search window indicates an apnea event when the present breathing volume is below a fraction K of the threshold breathing volume.

5. The method of claim 1, wherein the breathing signal is a breathing volume signal and wherein determining whether the change in volume of breathing of the patient within the apnea search period indicates an apnea event comprises:
analyzing the breathing volume signal over a first section of the apnea search period to calculate a present breathing volume;
comparing the present breathing volume to an absolute breathing volume for the patient; and
in response to determining that that present breathing volume is below the absolute breathing volume for the patient, determining occurrence of the apnea event.

6. The method of claim 5, wherein the absolute breathing volume for the patient is determined based on an age of the patient.

7. The method of claim 5, wherein the absolute breathing volume for the patient is determined based on a size of the patient.

8. The method of claim 5, wherein analyzing the breathing volume signal over the first section of the apnea search period to calculate the present breathing volume comprises:
analyzing the breathing volume signal over the first section of the apnea search period to determine an increase in the breathing volume signal indicating a short movement of the patient; and
adjusting the present breathing volume to discount for the increase in the breathing volume signal indicating the short movement of the patient.

9. A system comprising:
memory;
one or more processor units;
an ABD monitoring system stored in the memory and executable by the one or more processor units, the ABD monitoring system encoding computer-executable instructions on the memory for executing on the one or more processor units a computer process, the computer process comprising:
determining, based on an image signal received from a camera focused on at least a portion of a patient, a breathing volume signal;
determining an onset of a desaturation event for the patient and an onset of a bradycardia event for the patient;
determining length of a bradycardia-desaturation (BD) period between the onset of the desaturation event for the patient and the onset of the bradycardia event for the patient;
in response to determining that the length of the BD period is less than a BD time threshold, determining whether a change in volume of breathing of the patient within an apnea search period indicates an apnea event; and
in response to determining that the change in volume of breathing of the patient within the apnea search period indicates an apnea event, displaying an occurrence of an apnea-bradycardia-desaturation (ABD) event.

10. The system of claim 9, wherein determining the onset of the desaturation event for the patient comprising determining whether the oxygen saturation level of the patient is below 80%.

11. The system of claim 9, wherein determining the onset of the bradycardia event for the patient comprising determining whether the heart-rate signal of the patient is below 100 bpm.

12. The system of claim 9, wherein determining whether the change in volume of breathing of the patient within the apnea search period indicates an apnea event comprises:
analyzing the breathing volume signal over a first section of the apnea search period to calculate a present breathing volume;
analyzing the breathing volume signal over a preceding section of the apnea search period to calculate a threshold breathing volume, the preceding section being before the first section; and
determining that the change in volume of breathing of the patient within the apnea search window indicates an apnea event when the present breathing volume is below a fraction K of the threshold breathing volume.

13. The system of claim 9, wherein determining whether the change in volume of breathing of the patient within the apnea search period indicates an apnea event comprises:
analyzing the breathing volume signal over a first section of the apnea search period to calculate a present breathing volume;
comparing the present breathing volume to an absolute breathing volume for the patient; and
in response to determining that that present breathing volume is below the absolute breathing volume for the patient, determining occurrence of the apnea event.

14. The system of claim 13, wherein the absolute breathing volume for the patient is determined based on an age of the patient.

15. The system of claim 13, wherein the absolute breathing volume for the patient is determined based on a size of the patient.

16. A physical article of manufacture including one or more tangible computer-readable storage media encoding computer-executable instructions for executing on a computer system a computer process to provide an automated connection to a collaboration event for a computing device, the computer process comprising:
determining, based on an image signal received from a camera focused on at least a portion of a patient, a breathing flow signal;
determining an onset of a desaturation event for the patient and an onset of a bradycardia event for the patient;
determining length of a bradycardia-desaturation (BD) period between the onset of the desaturation event for the patient and the onset of the bradycardia event for the patient;
in response to determining that the length of the BD period is less than a BD time threshold, determining whether a change in flow of breathing of the patient within an apnea search period indicates an apnea event; and
in response to determining that the change in flow of breathing of the patient within the apnea search period indicates an apnea event, displaying an occurrence of an apnea-bradycardia-desaturation (ABD) event.

17. The physical article of manufacture of claim 16, wherein determining the onset of the desaturation event for the patient comprising determining whether the oxygen saturation level of the patient is below 80%.

18. The physical article of manufacture of claim 16, wherein determining the onset of the bradycardia event for the patient comprising determining whether the heart-rate signal of the patient is below 100 bpm.

19. The physical article of manufacture of claim 16, wherein determining whether the change in flow of breathing of the patient within the apnea search period indicates an apnea event comprises:
analyzing the breathing flow signal over a first section of the apnea search period to calculate a present breathing flow;
analyzing the breathing flow signal over a preceding section of the apnea search period to calculate a threshold breathing flow, the preceding section being before the first section; and
determining that the change in flow of breathing of the patient within the apnea search window indicates an apnea event when the present breathing flow is below a fraction K of the threshold breathing flow.

20. The physical article of manufacture of claim 16, wherein determining whether the change in flow of breathing of the patient within the apnea search period indicates an apnea event comprises:
analyzing the breathing flow signal over a first section of the apnea search period to calculate a present breathing flow;
comparing the present breathing flow to an absolute breathing flow for the patient; and
in response to determining that that present breathing flow is below the absolute breathing flow for the patient, determining occurrence of the apnea event.
